(19) United States
(12) Patent Application Publication
(10) Pub. No.: US 2008/0113015 A1
(43) Pub. Date: May 15, 2008

(54) Superloaded Liposomes for Drug Delivery

(75) Inventors: Hermann Katinger, Vienna (AT); Andreas Wagner, Baden (AT); Karola Vorauer-Uhl, Vienna (AT); Renate Kunert, Deutch-Wagram (AT); Stefanie Strobach, Vienna (AT)

Correspondence Address: ROTHWELL, FIGG, ERNST & MANBECK, P.C., 1425 K STREET, N.W., SUITE 800, WASHINGTON, DC, 20005, US

(73) Assignee: POLYMUN SCIENTIFIC IMMUNBIOLOGISCHE FORSCHUNG GMBH, Vienna (AT)

(21) Appl. No.: 11/569,514

(22) PCT Filed: May 24, 2005
(86) PCT No.: PCT/EP05/05577

§ 371 Date: Jun. 22, 2007

(30) Foreign Application Priority Data
May 24, 2004 (EP) 04012224.4

(51) Int. Cl. A61K 9/127; A61K 38/16; A61K 31/715; A61P 43/00

(52) U.S. Cl. 424/450; 514/8; 514/54

(57) ABSTRACT

The present invention relates to liposomes for drug delivery, wherein a liposome includes molecules of at least one desired drug distributed within an aqueous phase in the interior of the liposome and wherein the liposome further includes molecules of the same or of another drug attached to either or both sides of the liposomal membrane. More specifically, the invention relates to liposomes, wherein at least a part of the molecules of a desired drug bear a functional group that is reactive with a functional group present in at least one lipid fraction, and wherein the drug is covalently linked to the membrane lipids by chemical bonding, e.g. by ester bonding of a hydroxyl group of a lipid molecule and an acidic residue of the drug. In a preferred embodiment, the desired drug is a glycoprotein such as erythropoietin. The invention further relates to a method of manufacture of said liposomes and to pharmaceutical compositions containing them.

### CROSS REFERENCE TO RELATED APPLICATION

This application is a 35 U.S.C. § 371 National Phase Entry Application from PCT/EP2005/005577, filed May 24, 2005, and designating the United States.

### BACKGROUND OF THE INVENTION

1. Field of the Invention

The present invention relates to liposomes containing erythropoietin or another pharmaceutically active compound being a glycoprotein or having a glycoprotein moiety with sialic acid groups at the end of its glycosilation sites. The invention further relates to a method of making the liposomes, to compositions containing them and to methods of using said liposomes.

2. Technical Background

Erythropoietin (EPO) is a well known glycoprotein involved in the synthesis of red blood cells. Pharmaceutical compositions comprising EPO together with human serum albumin (HSA) as a carrier are known in the art. However, as the use of HSA obtained from natural sources always bears a potential threat of transmitting infectious diseases, particularly viral infections, attempts have been made to replace HSA as carrier for parenteral EPO formulations.

EP 0 937 456 A1 discloses a liposome-based parenteral composition comprising erythropoietin together with liposomes in an aqueous dispersion, wherein the glycoprotein is not substantially incorporated within the liposomes but is essentially present in the aqueous phase outside the liposomes. The liposomes are being made by injecting an ethanolic lipid phase into an aqueous buffer using high speed homogenisation for making liposomes of less than 1 μm in diameter.

The Japanese patent JP8231417 discloses another method of making a liposomal EPO-composition, wherein liposomes are prepared by reverse-phase evaporation to include EPO within the liposomes.

### SUMMARY OF THE INVENTION

While the methods known in the art may have certain advantages, it is the goal of the present invention to outperform the known compositions by providing liposomes that comprise a desired drug not only encapsulated in the interior, e.g. in the aqueous phase of the liposomes but also associated with or attached to the inside and/or outside of the liposomal membrane, resulting in liposomes loaded with a desired drug at an extent exceeding the usually or even theoretically obtainable load by passive drug inclusion, as predictable by numerical calulation based on the amounts of drug and lipid and the average size and/or total internal volume of the liposomes (“captured volume”). Thus the present invention relates to liposomes comprising molecules of at least one desired drug, wherein said drug molecules are present in an amount exceeding the drug load achieved by mere passive inclusion of said drug within the aqueous interior, i.e. without attachment of the drug to the membrane.

The desired drug is a pharmaceutically active compound which in a preferred embodiment is a glycoprotein or another bioactive compound having at least an oligosaccharide or polysaccharide moiety, wherein said glycoprotein or oligosaccharide or polysaccharide moiety comprises freely accessible, hence reactive siatic acid groups, e.g. at the end of its glycosilation sites.

Accordingly, it is an object of the invention to provide densely loaded liposomes wherein a desired drug is encapsulated within the liposomes and wherein a portion of the drug is also associated with or attached to the liposome membrane from the exterior, and typically also from the interior. In a preferred embodiment said drug is erythropoietin.

It is another object of the invention to provide a method of making the densely loaded liposomes.

It is yet another object of the invention to provide pharmaceutical compositions containing such densely loaded liposomes.

### DETAILED DESCRIPTION OF THE INVENTION

More specifically, in a first embodiment the present invention relates to a liposome for drug delivery, wherein the liposome comprises molecules of at least one desired drug distributed within an aqueous phase in the interior of the liposome and wherein the liposome further comprises molecules of the same or of another drug attached to either or both sides of the liposomal membrane. The attachment may be of a covalent or non-covalent nature, i.e. there may be a chemical bonding between drug and lipid or the attachment may be caused by adhesion forces other than a chemical bonding, including but not limited to van der Waals forces, hydrogen bridges, electrostatic forces, hydrophilic-hydrophobic interactions, affinity forces, and/or polar interactions and the like between drug and lipid molecules.

In another embodiment the invention relates to such a liposome, wherein at least a part of the drug molecules attached to the membrane are covalently linked to membrane lipids.

In a preferred embodiment the covalent linkage is a chemical bonding between a reactive functional group of a lipid and a functional group of the drug that is reactive with the functional group of the lipid. It is also preferred that the chemical bonding be of a nature such that it does not impede the intended physiological action of the drug upon administration to a human or animal body. In another preferred embodiment the chemical bonding is of nature such that it may be cleaved under the physiological conditions of a human or animal body upon administration.

Accordingly, in a specific embodiment the invention relates to such a liposome, wherein the functional group of the lipid is a hydroxyl group, which hydroxyl group may be a part of a polyvalent alcohol residue. Suitable polyvalent alcohol residues are sugar alcohol residues, particularly those naturally occurring in lipids such as glycerol residues and inositol residues. However, the lipid functional group providing for the attachment of a desired drug to the liposome membrane may also be a choline residue, such as, for example, in phosphatidyl choline (lecithin).

Where the lipid functional group is a hydroxyl group it is preferred that the reactive functional group of the drug is an acidic group, particularly an acidic group selected from the group consisting of a phosphoric acid residue, sulphuric acid residue, carbonic acid residue, and sialic acid residue. In that case the drug molecules or at least a part of the drug molecules that bear an acidic group will be covalently linked by ester bonding to at least a part of the lipid molecules that bear a hydroxyl group.

In another embodiment the invention relates to a liposome as described above, wherein the drug is a glycoprotein or has an oligosaccharide or polysaccharide moiety which glycoprotein or oligo- or polysaccharide moiety comprises at least one free reactive sialic acid group that is accessible for esterification by hydroxyl-containing lipids.

The lipid composition of the vesicle membrane of the liposomes typically comprises at least one lipid of natural or non-natural origin selected from the group consisting of phospholipids, glykolipids, ceramides, and derivatives of any one of these kinds of lipids.

Accordingly, in another specific embodiment the invention relates to an aforementioned liposome, wherein the liposome comprises phospholipids, particularly phospholipids selected from the group consisting of sphingophospholipids and glycerophospholipids, the sphingophospholipids comprising sphingomyelins and the glycerophospholipids comprising lecithins, kephalins, cardiolipins, phosphatidylinositols and phosphatidylinositol phosphates.

Where the liposomes comprise glykolipids instead of or in addition to phospholipids, said glykolipids are preferably selected from the group consisting of glykosphingolipids and glykoglycerolipids, the glykosphingolipids comprising cerebrosides, gangliosides, sulfatides, and the glykoglycerolipids comprising glykosylmonoglycerides and glykosyldiglycerides.

In a very specific embodiment, a liposome according to the present invention has a lipid composition, wherein the membrane comprises di-palmitoyl-phosphatidylcholine (DPPC), cholesterol, and egg phosphatidylglycerol (EPG) at a molar ratio of DPPC:cholesterol:EPG=7:2:1, it being understood that this ratio may of course be varied qualitatively and/or quantitatively, as the case may be.

In another specific embodiment the drug for delivery encapsulated in and attached to the liposomes is erythropoietin.

In another embodiment, the invention relates to pharmaceutical compositions comprising a drug-loaded liposome as defined above together with a pharmaceutically acceptable carrier. The pharmaceutical composition may be in any suitable liquid, semi-liquid or solid form for administration, particularly for parenteral administration, such as in the form of an injection solution, a nasal spray, an inhalation liquid, a cream, a gel, an ointment, a suppository, or a lotion, in order to allow for topic or systemic parenteral administration.

The present invention further relates to a method of manufacture of a drug-loaded liposome comprising molecules of at least one desired drug distributed within an aqueous phase in the interior of the liposome and further comprising molecules of the same or of another drug attached to either or both sides of the liposomal membrane, the method comprising:
providing a lipid phase in an organic solvent, wherein the lipid phase comprises at least one lipid fraction wherein each lipid molecule has at least one reactive functional group;
providing an aqueous phase comprising a buffer solution and dissolved therein at least one desired drug, wherein at least one drug has a reactive functional group capable of reacting with a functional group of the lipids;
feeding the lipid phase into the aqueous phase under conditions allowing for the formation of liposomes;
optionally circulating the aqueous phase in a loop and repeating step c) in order to increase the efficiency of drug uptake by the liposomes; and
harvesting drug-loaded liposomes,
   wherein at least a part of the drug molecules are incorporated within the liposomes while another part of the drug molecules is attached to either or both sides of the liposomal membrane.

It shall be understood that terms such as “reactive functional group” or “reacting with a functional group”, as used herein in connection with drugs and lipids, do not necessarily imply that the kind of reaction or reactivity be of a nature such that it results in a covalent chemical linkage but instead may also be of a nature such as to result in a more or less strong adhesion or attachment rather than a chemical linkage betweet lipid and drug, as outlined hereinbefore.

As mentioned above, the functional group of the lipids may be a hydroxyl group, particularly a hydroxyl group as a part of a polyvalent alcohol residue such as a sugar alcohol residue, preferably derived from naturally occurring sugar alcohols such as glycerol and inositol, or a choline group as in lecithins.

Also, as mentioned above, the reactive functional group of the drug may be an acidic group preferably selected from the group consisting of a phosphoric acid residue, sulfuric acid residue, carbonic acid residue, and sialic acid residue.

In one embodiment the method of manufacture comprises feeding the lipidic phase into the aqueous phase under conditions allowing for interaction, e.g. chemical reaction, of at least a part of said lipids carrying a reactive functional group with at least a part of said drug molecules bearing a reactive functional group.

Where the lipid functional group is a hydroxyl group and the drug functional group is an acidic residue, said conditions allowing for interaction and optionally chemical reaction comprise feeding the lipidic phase into the aqueous phase at a reaction temperature of about 25 to about 65° C. and at a pH value of the aqueous phase of about 6 to about 8, whereupon at least a part of said drug molecules bearing a reactive functional group is being attached, optionally covalently linked by esterification, to at least a part of said lipids having functional groups.

In a more specific embodiment the drug is a glycoprotein or has an oligosaccharide or polysaccharide moiety which glycoprotein or oligo- or polysaccharide moiety comprises at least one reactive sialic acid group.

In yet another specific embodiment of the method of manufacture the lipidic phase comprises at least one lipid of natural or non-natural origin selected from the group consisting of phospholipids, glykolipids, ceramides, and derivatives of these lipids, while in a most specific embodiment the lipid composition of the lipidic phase is adjusted to comprise DPPC, cholesterol, and EPG at a molar ratio of 7:2:1, it being understood that this ratio may of course be widely varied qualitatively and/or quantitatively, as the case may be.

It is preferred that the lipidic phase be fed into the aqueous phase under pressure and under essentially shear-free conditions using the cross-flow injection technique disclosed in WO 02/36257 that allows for immediate and spontaneous formation of liposomes under extremely mild conditions, i.e. without using shear-force creating high speed homogenizers or similar means for creating high turbulence for vesicle formation.

In yet another embodiment the invention relates to a method of manufacture, wherein prior to the step of vesicle formation at least a part of the lipid fraction, or the entire lipid fraction, that contains lipids having at least one reactive functional group per lipid molecule, is being reacted with at least a part of those drug molecules that bear a reactive functional group, in order to attach or even covalently link drug molecules to membrane lipids. This pre-reaction is carried out prior to vesicle formation. Accordingly, in one embodiment the invention relates to a method of manufacture, wherein at least a part of the lipids of the lipidic phase is being attached, optionally covalently linked, to at least a part of the molecules of a desired drug prior to feeding the lipidic phase into the aqueous phase.

In a further embodiment, the invention relates to a method of manufacture of a drug-loaded liposome comprising molecules of at least one desired drug distributed within an aqueous phase in the interior of the liposome and further comprising molecules of the same or of another drug attached to either or both sides of the liposomal membrane, the method comprising:
providing a lipidic phase in an organic solvent or as a dried film, wherein the lipidic phase comprises at least one lipid fraction wherein each lipid molecule has at least one reactive functional group;
providing a first aqueous phase comprising a buffer solution;
providing a second aqueous phase comprising a buffer solution and dissolved therein at least one desired drug, wherein at least one drug has a reactive functional group capable of attaching to or chemically reacting with a functional group of the lipids;
combining the lipidic phase with the first aqueous phase under conditions allowing for the formation of liposomes;
combining the liposomes formed with the second aqueous phase under conditions allowing for an uptake of at least a part of the drug molecules into the liposomes and allowing for interaction, optionally chemical reaction of at least a part of said lipids carrying a reactive functional group with at least a part of said drug molecules bearing a reactive functional group; and
harvesting drug-loaded liposomes,
   wherein at least a part of the drug molecules are incorporated within the liposomes while another part of the drug molecules is attached to either or both sides of the liposomal membrane.

Using this method it is preferred that the lipid functional group is a hydroxyl group and the drug functional group is an acidic residue and said conditions allowing for interaction, e.g. for a chemical reaction, comprise combining the liposomes with the second aqueous phase at a reaction temperature of about 25 to about 65° C. and at a pH value of the second aqueous phase of about 6 to about 8, and incubating the resulting liposome suspension until at least a part of said drug molecules bearing a reactive functional group is being attached, optionally covalently linked by esterification, to at least a part of said lipids having functional groups.

In a further embodiment said method comprises an additional procedural step, wherein during or after incubation the liposome suspension is subjected to a further treatment for enhancing drug uptake into the liposomes, such treatment preferably being selected from the group consisting of sonication, electro-poration, vortexing, and gradient-driven transmembrane diffusion.

The covalent linkage may be an ester bonding. A suitable drug for “super-loading” liposomes with drug for delivery is erythropoietin.

The densely loaded liposomes of the present invention allow to considerably lower the dosage of parenteral application forms such as injection solutions, nasal sprays, or topical application forms such as creams, gels, ointments or lotions. Depending on the nature of the desired drug for delivery, the present liposome compositions allow adjustment of administration regimes of drugs in a way such as to reduce or substantially avoid undesired side-effects in human or animal recipients. Moreover, the liposomal formulations according to the present invention significantly increase and prolong bioavailability of pharmaceutically active compounds that are readily susceptible to degradation or damage under physiological conditions inside a human or animal body.

In addition to “super-loading” liposomes with a desired drug, it is another outstanding advantage of the present invention to offer the possibility to prepare liposomes containing two or more different drugs at reasonable amounts, wherein at least one drug is covalently or non-covalently attached to the liposome membrane while at least one other drug is present in the aqueous phase within the liposomes. Thus, using the concept of the present invention will enable a person of ordinary skill in the art to specifically design liposomes for the simultaneous delivery of two or more different drugs or drug components, where such delivery may be needed or beneficial, such as e.g. in combination therapies.

The present liposomes are preferably being made using the cross-flow injection technique disclosed in WO 02/36257, the contents of which shall be incorporated herein by reference. In brief, the method is based on injecting an organic lipid phase, typically an ethanolic lipid phase, into an aqueous phase through a tiny opening in the wall of a lipid phase piping and an adjacent opening in the wall of a second piping connected with said lipid phase piping and carrying an aqueous phase. The peculiarity with this method is that at the place of injection, i.e. the cross-flow injection module or mixing chamber, the piping conveying the lipidic phase is firmly connected to the piping of the aqueous phase in a way such that the pipings are in liquid connection with each other through a common orifice allowing organic liquid to enter, under pressure and in the form of a spray mist, the stream of the aqueous phase passing by said common orifice. No shear-force producing elements are present at the area of liquid intersection, i.e. where the organic lipid stream dashes in an approximately right angle into the aqueous stream, and yet liposomes are spontaneously formed in extraordinary quality and vesicle size distribution. This extremely mild method allows for the use of oxidation- or temperature-sensitive lipids as well as for the encapsulation of sensitive drugs that would otherwise be damaged or rendered inactive by cavitational phenomena or local overheating due to shear forces, such as may be the case e.g. in a homogenizer.

In addition to the method of passive incorporation of drugs into liposomes the present invention goes on to also actively attach desired drugs to the liposomal membrane, which typically is a unilamellar bilayer. In order to achieve this goal, i.e. to actively attach or associate a pharmaceutically active compound to the liposomal membrane, it is preferred that the lipid composition of the membrane is specifically selected in accordance with the desired drug(s) for delivery, i.e. to comprise lipids capable of interacting with the molecules of the desired drug(s), such as phospholipids of natural or non-natural origin having headgroups of the glycerol or inositol type, and to comprise other lipids, particularly of natural origin, which have only low amounts of lipids having such a headgroup.

Suitable lipids comprise sphingosins and derivatives thereof, cerebrosides and derivatives thereof, ceramides and derivatives thereof.

For active association or attachment of a desired drug to the liposomal membrane it is preferred that the liposomes be prepared in the presence of the desired drug, for instance erythropoietin, at a temperature of about 25 to about 65° C. and at a pH around neutral, i.e. at a slightly basic or slightly acidic pH, preferably at a pH of from 6 to 8. The attachment, optionally including chemical linkage by esterification reaction between the sialic acid groups and the phospholipids, appears to work best at these temperature and pH ranges.

The liposomes according to the present invention typically comprise one lipid fraction selected from the group consisting of di-palmitoyl phosphatidylcholine (DPPC), egg phosphatidylcholine (EPC), soy phosphatidylcholine (SPC), ceramides and sphingosins as the main lipid fraction, together with at least one other lipid fraction selected from the group consisting of cholesterol and lipids having charged headgroups.

Using the aforementioned method of passive encapsulation by cross-flow injection of the lipid phase into the aqueous phase the following results were obtained:
a) by passive encapsulation
encapsulation rates of up to 20-50% by weight of the drug subjected to encapsulation were achieved, the results primarily depending on
   the number of recirculation cycles performed with the drug-containing aqueous phase,
   the preselected vesicle size of the developing liposomes, and
   the lipid composition and lipid concentration of the membrane.

Such encapsulation rates are well in accordance with results from previous studies, e.g. for the liposomal inclusion of certain enzymes such as rhSOD; whereas
b) by a combined passive and active loading of the liposomes according to the present invention
loading rates, i.e. encapsulation plus interior and exterior attachment to the liposomal membrane, of up to 100% wt of the drug provided for loading were achieved, as confirmed by analysis of the filtrates after liposome loading. Typically, free drug was found in the filtrates in a concentration of 10% wt or less, relative to the orginally supplied concentration.

Most of the experiments that have led to the present invention were carried out using erythropoietin as a desired drug to exemplify the drug-loading concept of the present invention. However, as mentioned hereinbefore the invention is applicable to any substances that may be both encapsulated within liposomes and attached to the liposomal membrane, preferably on either or both sides of the membrane. The attachment may be of a covalent or non-covalent nature, for instance an attachment by affinity, electrostatic interaction, van der Waals forces, hydrophilic and/or hydrophobic interaction, and the like, as known in the art. It is preferred, however, that the drug be covalently linked to a functional group of a lipid contained in the liposomal membrane. Where the functional group is of a polyol type, particularly of a sugar alcohol type, and typically is either inositol or glycerol, a desired drug will be most efficiently attached to the membrane if it contains a reactive acidic functional group such as a phosphoric acid, sulphuric acid, carbonic acid or sialic acid residue. Such an acidic reactive functional group may upon contact with a hydroxyl group of the lipid cause a covalent bonding between the lipid and the drug through esterification.

In order that the invention herein described be more fully understood, the subsequent examples are set forth.

### EXAMPLE 1 (COMPARATIVE EXAMPLE)

### Passive Loading of Liposomes With Drug

a) Lipid composition
DPPC:cholesterol:stearylamine=7:2:1
   b) Drug concentration
2868 μg erythropoietin (EPO) per 2 ml aqueous buffer (PBS)

The liposomes were manufactured according to the cross-flow injection method disclosed in WO 02/36257. Unilamellar bilayer liposomes having a mean vesicle diameter of about 170 nm were obtained and subsequently subjected to a filtration step using a ultra/diafiltration unit.

The filtrate was analyzed for free, i.e. non-encapsulated, drug the results being as follows:
liposome retentate (2 ml): 711 μg EPO=25% wt encapsulation;
total filtrates: 2175 μg EPO=75% wt non-encapsulated;
   broken down into fractions: filtrate F1=843 μg; filtrate F2=596 μg, filtrate F3=345 μg, filtrate F4=391 μg.

In this example the use of the state-of-the art method for encapsulation of EPO within liposomes resulted in 25% wt inclusion of the drug, e.g. 356 μg drug per 1 ml liposome retentate. More generally, using this method of passive encapsulation of drugs typically resulted in inclusion rates ranging from about 25 to about 50% by weight, relative to the initially provided amount of drug.

This loading efficiency is well in line with previous studies using peptides or proteins such as recombinant human superoxide dismutase (rhSOD) as desired drugs for liposomal encapsulation.

### EXAMPLE 2

a) Lipid composition
DPPC:cholesterol:EPG=7:2:1
   b) Drug concentration
3375 μg Epo in 2 ml aqueous buffer (PBS)

The liposomes were manufactured according to the cross-flow injection method disclosed in WO 02/36257. Unilamellar bilayer liposomes having a mean vesicle diameter of about 170 nm were obtained and subsequently subjected to a filtration step using a ultra/diafiltration unit.

The filtrate was analyzed for free, i.e. non-encapsulated drug the results being as follows:
liposome retentate (2 ml): 3043 μg EPO=90% wt
total filtrates: 328 μg EPO=10% wt;
   broken down into fractions: filtrate F1=24 μg; filtrate F2=128 μg,
      filtrate F3=0-1 μg, filtrate F4=176 μg.

In this example the cross-flow injection method for encapsulation of EPO within liposomes was carried out at a pH of 7.5 and at a temperature of 50° C., which resulted in 90% inclusion of the drug, corresponding to 1522 μg drug per 1 ml liposome retentate.

More generally, using this or an equivalent method of combined passive and active loading of liposomes with desired drugs by proper selection of lipids that allow for ready attachment, optionally involving covalent bonding of the drug to the liposome membrane (in addition to passive inclusion of the drug within the aqueous interior of the liposomes) will typically result in loading rates ranging from about 80 to about 100% by weight, relative to the initially provided amount of drug.

### EXAMPLE 3

### Combination of Different Drugs

Example 2 was repeated except that the aqueous phase further contained 3500 μg of rhSOD. After filtration, the liposome retentate comprised about 25% (approx. 820 μg per 2 ml retentate) of the rhSOD and about 70% (approx. 2200 μg per 2 ml retentate) of EPO, while the balance of the amounts of drug was detected in the filtrates.

This example demonstrates that it is possible to “superload” liposomes even when using two different drugs at the same time. While the internal aqueous space of the liposomes was able to absorb, as expected, around 25% wt of the total rhSOD offered, the liposomes were also able to absorb large amounts (approx. 70% wt) of the second drug, i.e. EPO. This was possible due to the interactions between the second drug and the EPG lipid fraction of the liposome membrane resulting in an attachment of said drug to said lipid fraction in the liposome membrane, plus a certain extent of passive inclusion within the aqueous interior of the liposome vesicles (not separately quantified herein).

The experiments described herein and further experiments (data not shown) impressingly demonstrated the superiority of the kind of active loading according to the present invention over conventional passive inclusion of a drug-containing aqueous phase. In fact, the present method of active loading and, particularly, of combined active and passive loading yielded liposomes loaded with drug in an amount of up to 200% of the amounts that could be theoretically expected according to the captured volume of aqueous phase.

The highly concentrated liposome suspension harvested from the aqueous phase according to the present invention typically comprises about 0.5-2 mg drug per ml. It may be subsequently rediluted and/or formulated into usual solid, liquid or semi-liquid galenic preparations and pharmaceutical compositions for oral or parenteral application, particularly for intramuscular, intravenous, subcutaneous, cutaneous, intramucosal, intranasal, or intrapulmonary administration. Suitable compositions may be in the form of injection solutions, nasal sprays, inhalation liquids, creams, gels, ointments, suppositories, lotions, and the like, for topical or systemic administration.

We claim:

1-34. (canceled)

35. Liposome for drug delivery that comprises molecules of at least one desired drug distributed within an aqueous phase in the interior of the liposome, characterized in that it further comprises molecules of the same or of another drug attached to either or both sides of the liposomal membrane.

36. Liposome according to claim 35, wherein said drug molecules are present in an amount exceeding a maximum drug load achievable by passive inclusion of said drug within the aqueous interior without attachment of drug molecules to the membrane.

37. The liposome according to claim 35, wherein at least a part of the drug molecules is attached to the membrane through covalent linkage by chemical bonding or through non-covalent attachment by adhesion forces other than a chemical bonding, typically including at least one kind of adhesion forces selected from the group consisting of van der Waals forces, hydrogen bridges electrostatic forces, hydrophilic-hydrophobic interactions, affinity forces, and polar interactions, between drug and lipid molecules.

38. The liposome according to claim 35, wherein the attachment is between a reactive functional group of a lipid and a functional group of the drug.

39. Liposome according to claim 38, wherein the functional group of the lipid is a hydroxyl or a choline group.

40. Liposome according to claim 39, wherein the hydroxyl group is part of a polyvalent alcohol residue.

41. Liposome according to claim 40, wherein the polyvalent alcohol residue is a sugar alcohol residue selected from the group consisting of a glycerol residue and an inositol residue.

42. Liposome according to claim 37, wherein the reactive functional group of the drug is an acidic group selected from the group consisting of a phosphoric acid residue, sulphuric acid residue, carbonic acid residue, and sialic acid residue, and at least a part of the drug being attached to membrane lipids, optionally covalently linked to the lipids by an ester bonding.

43. Liposome according to claim 42, wherein the drug is a glycoprotein or has an oligosaccharide or polysaccharide moiety, which glycoprotein or oligo- or polysaccharide moiety comprises at least one reactive sialic acid group.

44. Liposome according to claim 35, wherein the membrane comprises at least one lipid of natural or non-natural origin selected from the group consisting of phospholipids, glykolipids, ceramides, and derivatives of these lipids.

45. Liposome according to claim 44, wherein the phospholipids are selected from the group consisting of sphingophospholipids and glycerophospholipids, the sphingophospholipids comprising sphingomyelins and the glycerophospholipids comprising lecithins, kephalins, cardiolipins, phosphatidylinositols and phosphatidylinositol phosphates.

46. Liposome according to claim 44, wherein the glykolipids are selected from the group consisting of glykosphingolipids and glykoglycerolipids, the glykosphingolipids comprising cerebrosides, gangliosides, sulfatides, and the glykoglycerolipids comprising glykosylmonoglycerides and glykosyldiglycerides.

47. Liposome according to claim 35, wherein the membrane comprises DPPC, cholesterol, and EPG at a molar ratio of 7:2:1.

48. Liposome according to claim 35, wherein the drug is erythropoietin.

49. Pharmaceutical composition comprising a drug-loaded liposome defined in claim 35, together with a pharmaceutically acceptable carrier for oral or parenteral administration.

50. The pharmaceutical composition according to claim 49, in the form of an injection solution, a nasal spray, an inhalation liquid, a cream, a gel, an ointment, a suppository, or a lotion.

51. The pharmaceutical composition according to claim 49, for topic or systemic parenteral administration.

52. A method of manufacture of a drug-loaded liposome comprising molecules of at least one desired drug distributed within an aqueous phase in the interior of the liposome and further comprising molecules of the same or of another drug attached to either or both sides of the liposomal membrane, the method comprising:
providing a lipidic phase in an organic solvent, wherein the lipidic phase comprises at least one lipid fraction wherein each lipid molecule has at least one reactive functional group;
providing an aqueous phase comprising a buffer solution and dissolved therein at least one desired drug, wherein at least one drug has a reactive functional group capable of attaching to or chemically reacting with a functional group of the lipids;
feeding the lipidic phase into the aqueous phase under conditions allowing for the formation of liposomes;
optionally circulating the aqueous phase in a loop and repeating the feeding step in order to increase the efficiency of drug uptake by the liposomes; and
harvesting drug-loaded liposomes,
wherein at least a part of the drug molecules are incorporated within the liposomes while another part of the drug molecules is attached to either or both sides of the liposomal membrane.

53. The method according to claim 52, wherein the functional group of the lipids is a hydroxyl or a choline group.

54. The method according to claim 53, wherein the hydroxyl group is part of a polyvalent alcohol residue.

55. The method according to claim 54, wherein the polyvalent alcohol residue is a sugar alcohol residue selected from the group consisting of a glycerol residue and an inositol residue.

56. The method according to claim 52, wherein the reactive functional group of the drug is an acidic group selected from the group consisting of a phosphoric acid residue, sulphuric acid residue, carbonic acid residue, and sialic acid residue.

57. The method according to claim 52, wherein the lipidic phase is fed into the aqueous phase under conditions allowing for interaction of at least a part of said lipids carrying a reactive functional group with at least a part of said drug molecules bearing a reactive functional group.

58. The method of claim 57, wherein the lipid functional group is a hydroxyl group and the drug functional group is an acidic residue and wherein said conditions allowing for interaction comprise feeding the lipidic phase into the aqueous phase at a reaction temperature of 25 to 65° C. and at a pH value of the aqueous phase of 6 to 8, whereupon at least a part of said drug molecules bearing a reactive functional group is being attached, optionally covalently linked by esterification, to at least a part of said lipids having functional groups.

59. A method of manufacture of a drug-loaded liposome comprising molecules of at least one desired drug distributed within an aqueous phase in the interior of the liposome and further comprising molecules of the same or of another drug attached to either or both sides of the liposomal membrane, the method comprising:
providing a lipidic phase in an organic solvent or as a dried film, wherein the lipidic phase comprises at least one lipid fraction wherein each lipid molecule has at least one reactive functional group;
providing a first aqueous phase comprising a buffer solution;
providing a second aqueous phase comprising a buffer solution and dissolved therein at least one desired drug, wherein at least one drug has a reactive functional group capable of attaching to or chemically reacting with a functional group of the lipids;
combining the lipidic phase with the first aqueous phase under conditions allowing for the formation of liposomes;
combining the liposomes formed with the second aqueous phase under conditions allowing for an uptake of at least a part of the drug molecules into the liposomes and allowing for interaction, optionally chemical reaction, of at least a part of said lipids carrying a reactive functional group with at least a part of said drug molecules bearing a reactive functional group; and
harvesting drug-loaded liposomes,
wherein at least a part of the drug molecules are incorporated within the aqueous interior of the liposomes while another part of the drug molecules is attached to either or both sides of the liposomal membrane.

60. The method of claim 59, wherein the lipid functional group is a hydroxyl group and the drug functional group is an acidic residue and wherein said conditions allowing for interaction, optionally chemical reaction, comprise combining the liposomes with the second aqueous phase at a reaction temperature of 25 to 65° C. and at a pH value of the second aqueous phase of 6 to 8, and incubating the resulting liposome suspension until at least a part of said drug molecules bearing a reactive functional group is being attached, optionally covalently linked by esterification, to at least a part of said lipids having functional groups.

61. The method of claim 60, wherein during or after incubation the liposome suspension is subjected to a further treatment for enhancing drug uptake into the liposomes, such treatment preferably being selected from the group consisting of sonication, electroporation, vortexing, and gradient-driven transmembrane diffusion.

62. The method according to claim 52, wherein the drug is a glycoprotein or has an oligosaccharide or polysaccharide moiety, which glycoprotein or oligo- or polysaccharide moiety comprises at least one reactive sialic acid group.

63. The method according to claim 52, wherein the lipidic phase comprises at least one lipid of natural or non-natural origin selected from the group consisting of phospholipids, glykolipids, ceramides, and derivatives of these lipids.

64. The method according to claim 52, wherein the lipidic phase comprises DPPC, cholesterol, and EPG at a ratio of 7:2:1.

65. The method according to claim 52, wherein the lipidic phase is fed into the aqueous phase under pressure and essentially shear-free conditions using a cross-flow injection technique that allows for immediate and spontaneous formation of liposomes.

66. The method according to claim 52, wherein at least a part of the lipids of the lipidic phase is being attached, optionally covalently linked, to at least a part of the molecules of the desired drug prior to feeding the lipidic phase into the aqueous phase.

67. The method according to claim 66, wherein the covalent linkage is an ester bonding.

68. The method according to claim 52, wherein the desired drug is erythropoietin.
